(19) United States
(12) Patent Application Publication
(10) Pub. No.: US 2025/0205507 A1
(43) Pub. Date: Jun. 26, 2025

(54) AN EYE MASK FOR PROVIDING A THERAPY TO EYES

(71) Applicant: Shenzhen Kaiyan Medical Equipment Co., Ltd., Shenzhen (CN)

(72) Inventors: Alain Dijkstra, Amstelveen (NL); LI XIANG, Shenzhen (CN); Hu Chunlei, Shenzhen (CN); Zhang Yong, Shenzhen (CN)

(73) Assignee: Shenzhen Kaiyan Medical Equipment Co., Ltd., Shenzhen (CN)

(21) Appl. No.: 18/967,714

(22) Filed: Dec. 4, 2024

(30) Foreign Application Priority Data
Dec. 20, 2023 (CN) 202323499016.5

(51) Int. Cl. A61N 5/06; A61F 9/00; A61N 1/04; A61N 5/00

(52) U.S. Cl. CPC A61N 5/06; A61F 9/0026; A61N 1/0456; A61N 1/0484; A61N 2005/005; A61N 2005/0648; A61N 2005/0652

(57) ABSTRACT

Embodiments of the present invention provide an eye mask for providing a therapy to eyes of a user. The eye mask comprises an inner layer, an outer layer and a circuit board. The inner layer has a first depression cavity and a second depression cavity such that the periphery of the first depression cavity and the second depression cavity form a convex edge. The circuit board comprises a plurality of LEDs for providing photo therapy and a micro-current element in arranged on the inner layer for providing micro-current therapy around the eyes of the user. A protective layer is provided over the LEDs to prevent direct exposure of eyes with the light emitted by the plurality of LEDs.

### TECHNICAL FIELD

The present invention generally relates to a therapeutic devices and, more particularly, to an eye mask that provide therapeutic effects to a user.

### BACKGROUND ART

Light therapy has a variety of effects on human biological activities. Red light to near-infrared light helps to promote the regeneration of collagen and skin cells and other tissues. The light therapy can be used in anti-wrinkle treatment, beauty effect, promote wound healing, freckle and scar removal and other fields. The irradiation and stimulation of the scalp by light can also affect the activity of brain cells, and then play a variety of therapeutic and physiotherapy roles.

The light therapy has also been proved beneficial in treating certain eye related disorder. The conventional device available for light therapy generally comprises an intelligent eye shield that comprises an outer appearance layer, an intermediate flexible circuit layer, an inner layer of protection isolation layer, the appearance layer is used as the outer surface layer of the eye mask. A circuit of a photoelectric generator is arranged on the flexible circuit layer, the photoelectric generator comprises a chip controller, a power supply and a switch arranged on the control board. The photoelectric generator is also provided with a lighting circuit that is connected to a plurality of LED light sources, and an electrical stimulator circuit that is connected to a plurality of point-shaped electrodes is arranged. The chip controller controls the working state of the illumination circuit and the electrical stimulator circuit.

The conventional devices used for phototherapy on eyes has several disadvantages, such that when the device is used for phototherapy on eyes, the electrodes are not set in an appropriate position that may cause irreparable damage to the eyes causing damage to the eyeball. Moreover, the light emitted by the LEDs directly fall on the eyes of the user that may also impair the vision of the user.

In order to overcome the disadvantage associated with the prior art, the present invention provides an eye mask that can be worn comfortably by a user and the eye mask does not cause any damage to the eyeball.

### OBJECTS OF THE INVENTION

Some of the objects of the invention are as follows:

An object of the present invention is to provide an eye mask that can be worn comfortably by a user.

Another object of the present invention is to provide an eye mask that can provide phototherapy to the user eye without damaging the eyeball of the user.

Another object of the present invention is to provide an eye mask that shield the eyes of the user from direct exposure of light emitted by the light source.

Another object of the present invention is to provide an eye mask that can provide micro-current therapy along with the phototherapy to the user.

Another object of the present invention is to provide an eye mask that can store and dispense medicament to improve the effect of micro-current therapy.

Another object of the present invention is to provide an eye mask that can provide heating or cooling therapy along with phototherapy and micro-current therapy.

### SUMMARY OF THE INVENTION

According to a first aspect of the present invention, an eye mask for providing a therapy to eyes is provided. The eye mask comprising: an inner layer, the inner layer is provided with a first depression cavity corresponding to a left eye of a user and a second depression cavity corresponding to a right eye of the user; an outer layer; a circuit board arranged between the inner layer and the outer layer; a plurality of LEDs arranged on the circuit board to target the left eye and the right eye of the user, such that the plurality of LEDs are positioned over the first depression cavity and the second depression cavity; wherein the first depression cavity and the second depression cavity creates a gap between the plurality of LEDs and eyes of the user; a protective layer arranged in the first depression cavity and the second depression cavity to protect the left eye and the right eye of the user from direct exposure to light emitted by the plurality of LEDs.

In one embodiment of the invention, the first depression cavity and the second depression cavity are concave in the direction of the outer layer.

In one embodiment of the invention, periphery of the first depression cavity and the second depression cavity forms a convex edge.

In one embodiment of the invention, the convex edge comprises an embedded liquid flow channel to store a liquid around the convex edge.

In one embodiment of the invention, the eye mask further comprises a heating element or a cooling element in contact with the liquid in the embedded liquid channel to heat or cool the liquid.

In one embodiment of the invention, the protective layer is made of a fabric or textile material.

In one embodiment of the invention, the first depression cavity and the second depression cavity in the inner layer is provided with a plurality of holes for the plurality of LEDs to pass through the plurality of holes and the plurality of holes allow the plurality of LEDs to position over the first depression cavity and the second depression cavity.

In one embodiment of the invention, the eye mask further comprising a plurality of massage head for providing massage therapy around the eyes of the user.

According to a second aspect of the present invention, an eye mask for providing a therapy to eyes is provided. The eye mask comprising: an inner layer, the inner layer is provided with a first depression cavity corresponding to a left eye of a user and a second depression cavity corresponding to a right eye of the user; an outer layer; a circuit board arranged between the inner layer and the outer layer; a plurality of LEDs arranged on the circuit board to target the left eye and the right eye of the user, such that the plurality of LEDs are positioned over the first depression cavity and the second depression cavity; wherein the first depression cavity and the second depression cavity creates a gap between the plurality of LEDs and eyes of the user; a micro-current element arranged between the inner layer and the circuit board, the micro-current element is electrically connected with the circuit board, wherein electrodes of the micro-current element are arranged on the periphery of the first depression cavity and the second depression cavity to provide micro-current at orbit around eyes of the user; a protective layer arranged in the first depression cavity and the second depression cavity to protect the left eye and the right eye of the user from direct exposure to light emitted by the plurality of LEDs.

In one embodiment of the invention, the first depression cavity and the second depression cavity are concave in the direction of the outer layer and periphery of the first depression cavity and the second depression cavity forms a convex edge.

In one embodiment of the invention, the convex edge comprises an embedded liquid flow channel to store a liquid around the convex edge.

In one embodiment of the invention, the eye mask further comprises a heating element or a cooling element in contact with the liquid the embedded liquid channel to heat or cool the liquid.

In one embodiment of the invention, the eye mask further comprises a plurality of massage head on the convex edge to provide massage therapy around the eyes of the user.

In one embodiment of the invention, the micro-current comprises a conductive cloth and a conductive silica gel, the conductive silica gel is arranged on the conductive cloth.

In one embodiment of the invention, the at least one contact surface of the micro-current element is a conductive protrusion, the conductive protrusion is located on the conductive silica gel, and the conductive protrusion passes through the first through hole.

In one embodiment of the invention, the protective layer is made of a fabric or textile material.

In one embodiment of the invention, the first depression cavity and the second depression cavity in the inner layer is provided with a plurality of holes for the plurality of LEDs to pass through the plurality of holes.

In one embodiment of the invention, the position of the plurality of LEDs on the circuit board is corresponding to the first depression cavity and the second depression cavity, and the plurality of holes allow the plurality of LEDs to positioned on the first depression cavity and the second depression cavity.

In one embodiment of the invention, the convex edge further comprises a plurality of pores for dispensing a medicament over surface of eyes of the user.

According to a third aspect of the present invention, a method for providing a therapy to eyes is provided. The method comprising: providing a phototherapy to eyes by a plurality of LEDs positioned in an eye mask; avoiding direct contact of the plurality of LEDs with eye socket by arranging the plurality of LEDs in a first depression cavity and a second depression cavity in an inner layer of the eye mask; providing a protective layer in the first depression cavity and the second depression cavity to prevent direct exposure of light emitted by the plurality of LEDs with eyes of the user; providing a micro-current therapy to eyes of the user using a micro-current element provided in the eye mask.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of the specification, the phrase “communication network” refers to a group of several connected devices including computing devices (such as desktops, mobile handheld devices, tablet PCs, notebooks, etc.), local and remotely located servers (such as web servers, application servers, database servers, Application Program Interface (API) servers, load balancers, compute nodes, and the like), routers, antennas, modems, multiplexers, demultiplexers, and the like. In that regard, the aforementioned connected devices may be able to exchange data signals through wired and/or wireless means as per several combinations of several different communication protocols such as 802.11 (Wi-Fi), 802.3 (Ethernet), Bluetooth, NFC, ZigBee and 3GPP protocols such as HSPA, HSDPA, LTE, GSM, CDMA, WLL and the like.

In the context of the specification, when an element is referred to as being “fixed to” or “disposed to” another element, it may either directly on another element or indirectly on that other element. When a component is said to be “connected” or “connected to” another component, it may be directly connected to another component or indirectly connected to other component on the piece.

In the context of the specification, the terms “first”, “second” and “third” are only used for descriptive purpose and does not implicate the relative importance or to implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to Nth Degree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 shows a perspective view of an eye mask for providing therapy to a user in accordance with an embodiment of the present invention.

FIG. 2 shows a back view of the eye mask in accordance with an embodiment of the present invention.

FIG. 3 shows an exploded view of the eye mask in accordance with an embodiment of the present invention.

FIG. 4 shows a top view of the eye mask in accordance with an embodiment of the present invention.

FIG. 5 shows a front view of the eye mask in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose an eye mask for providing therapeutic treatment to a user's eye. The eye mask comprises an inner layer and an outer layer. A circuit board is arranged between the inner layer and the outer layer and a plurality of LEDs are arranged on one side of the circuit board facing the inner layer. The eye mask further comprises a battery arranged on the circuit board, and the circuit board is electrically connected with the battery. The inner layer is provided with a first depression cavity that corresponds to a left eye of a user and a second depression cavity that corresponds to a right eye of a user. The first depression cavity and the second depression cavity are concave in the direction of the outer layer. The periphery of the concave firs depression cavity and the second depression cavity is correspondingly formed as a convex edge. A micro-current clement is arranged between the inner layer and the circuit board, the micro-current element is electrically connected with the circuit board. The micro-current element comprises at least one electrical contact surface. The convex edge are provided with one or more through holes and the electric contact surface is in contact with the human eye socket through the one or more through holes. The first depression cavity and the second depression cavity are provided with a protective layer and the protective layer is positioned over the plurality of LEDs to protect the direct exposure of the user's eye with light emitted from the plurality of LEDs. The setting of the first depression cavity and the second depression cavity prevents the plurality of LEDs to directly contact the eyeball and thus preventing injuries to the eyes when the light is too bright. The setting of at least one electric contact surface of the micro-current element can effectively treat the periocular area.

Several additional functionalities may also be added to the therapeutic device. For example, the inner layer of eye mask is provided with an embedded liquid channel that circulate a liquid which is connected with a heating or cooling element to provide heating or cooling effect around the eyes of the user. The eye mask further comprises a liquid medicament to augment the effect of micro-current therapy. The liquid medicament can be dispensed through a plurality of pores provided on the convex edge. The device can further comprise a control circuit board, the control circuit board is provided with a button and a controller cover and the outer layer is provided with a protruding part to accommodate the controller cover.

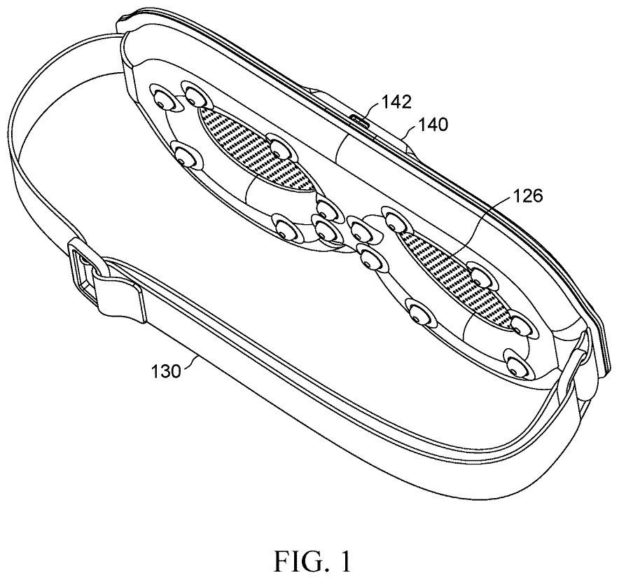

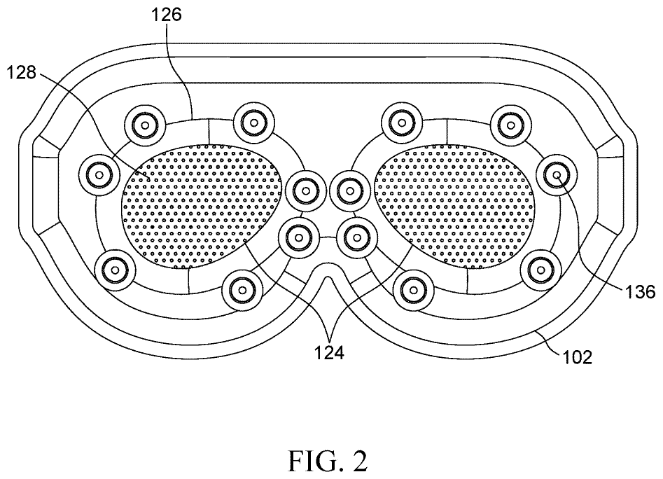

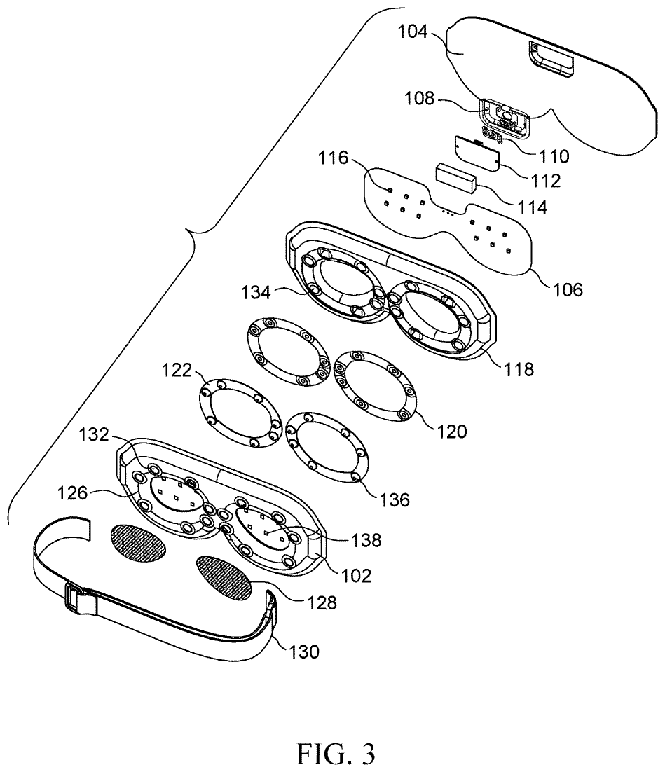

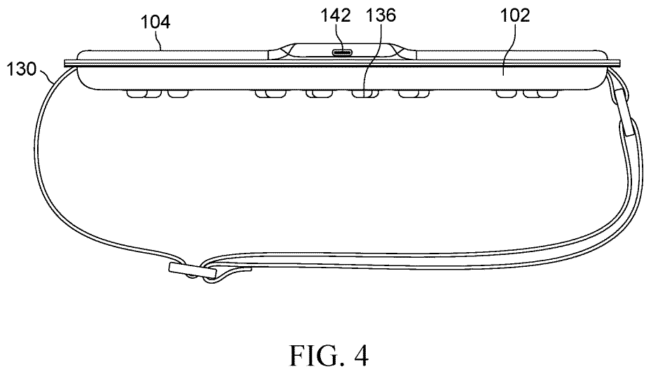

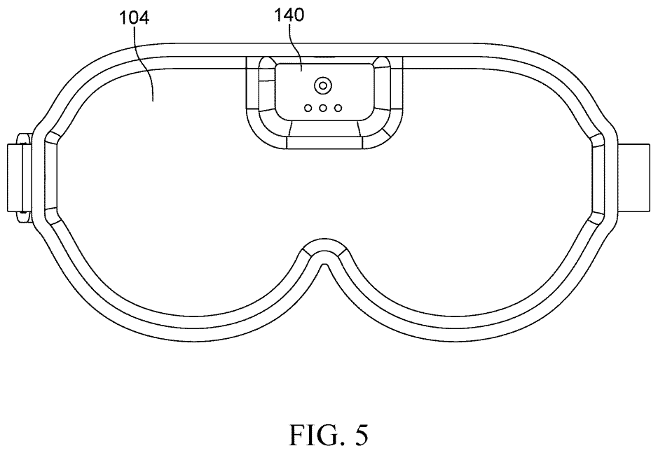

Referring to FIG. 1 to FIG. 5, the eye mask comprises an inner layer 102 and an outer layer 104. A circuit board 106 is arranged between the inner layer 102 and the outer layer 104. A plurality of LEDs 116 are arranged on one side of the circuit board 106 towards the inner layer 102. A battery 114 is further arranged on the circuit board 106, and the circuit board 106 is electrically connected with the battery 114. In the middle of the inner layer 102, two depression cavities 124 are arranged in the direction of the outer layer 104 so that the periphery of the depression cavity 124 is correspondingly formed as a convex edge 126. The two depression cavities 124 include a first depression cavity that corresponds to a left eye of a user and a second depression cavity that corresponds to a right eye of the user. The function of the depression cavities 124 is to improve the comfort of user and avoid the eye mask to compress the eyeball. The depression cavities 124 ensure that the plurality of LEDs 116 and the eyeball have a certain distance to achieve better treatment effect. When the eye mask is worn by the user, the convex edge 126 covers the skin around the user's eyes. The eye mask is provided with a pair of slots at the opposite sides. Two ends of a strap 130 fixed into the pair of slits. The strap can be worn around the user's head to position the eye mask over the eyes of the user.

The eye mask also has a micro-current element present between the inner layer 102 and the circuit board 106. The micro-current element is electrically connected with the circuit board 106. The micro-current element comprises at least one electrical surface contact in form of an electrode. On the convex edge 126, a first through hole 132 is arranged, and the electric contact surface of the micro-current clement passes through the first through hole 132. Because the human eyeball is relatively fragile and it is not advisable to use micro-current therapy on the eyeball, the micro-current element is arranged on the convex edge 126 and is used for electrode treatment of the orbit around the eyes.

Electrode therapy stands for Transcutaneous Electrical Nerve Stimulation Therapy (Tens), and the parts used to deliver electrical current and come into contact with the skin are often referred to as “electrical contacts.” These electrical contacts are an important part of Tens devices and are used to deliver a specific waveform of electrical current to the surface of the body to provide pain relief or healing effects. Electrical contacts come in different shapes and sizes, and can be small patches attached to the skin, patches, or patch contacts with wire connections. They are usually made of materials with good conductivity, such as carbon, hydrogels, conductive fabrics, etc.

The circuit board 106 is provided with a flexible layer 118 close to one side of the inner layer 102. The micro-current element is arranged on the flexible layer 118 and the flexible layer 118 is provided with a second through hole 134. The micro-current element is electrically connected with the circuit board 106 through the second through hole 134. The flexible layer 118 can be made up of a material such as sponge or flexible rubber. These materials increase the user's comfort and prevent the electrical contact from compressing the user's eye area.

The micro-current element is composed of a conductive cloth 120 and a conductive silica gel 122. The conductive silica gel 122 is arranged on the conductive cloth 120. Conductive silicone is soft, flexible, and has good electrical conductivity. The conductive silica gel 122 and the conductive cloth 120 are connected by an adhesive or glue.

The electrical contact surface of the micro-current element is a conductive protrusion 136. The conductive protrusion 136 is located on the conductive silica gel 122, and the conductive protrusion 136 passes through the first through hole 132. When used, the conductive protrusion 136 can be applied to the body's eye area, which may help relieve eye pain, headaches, tension headaches, or eye muscle spasms.

In an embodiment of the present invention, the inner layer 102 is a transparent inner layer. The transparent inner layer enables the light of the plurality of LEDs 116 or light therapy lamp bead to pass through the inner layer 102 to the eye. Phototherapy can be used to treat eye diseases such as dry eye, blepharitis, vitreous opacity, retinopathy, etc., and promote tissue repair and blood circulation through specific wavelengths of light stimulation, reduce eye inflammation, relieve pain, and promote the healing and health of eye tissues.

In another embodiment of the present invention, the inner layer 102 is provided with a plurality of holes 138. The plurality of LEDs pass through the plurality of holes 138. The inner layer 102 does not have to be a light-transmitting material. The position of the plurality of LEDs 116 on the circuit board 106 corresponds to the position of the first depression cavity and the second depression cavity. The plurality of holes 138 are arranged in the first depression cavity and the second depression cavity. The first depression cavity and the second depression cavity are provided with a protective layer 128. The protective layer 128 is positioned over the plurality of LEDs 116. The protective layer 128 can prevent the plurality of LEDs 116 from scratching the eyes. The material used for protective layer 128 is a fabric or textile material, such as acrylic, polyester, nylon etc. In an embodiment, the protective layer 128 is a filter layer which is adapted to block the UV rays or control the intensity outcome. The filter layer can have a filtering structure for achieving filtering of a specific wavelength and an expression color.

In an embodiment of the present invention, the eye mask of the present invention is provided with one or more stimulation elements, such as a heating element or a cooling element. The convex edge 126 has a liquid circulating channel that stores a liquid around the orbit of the two depression cavity 124 (the first depression cavity and the second depression cavity). The liquid circulating channel is in contact with the heating element and the cooling element. When the heating clement is used, the heating element heat up the liquid in the liquid circulating channel and orbit around the eye socket of the user is provided with the heat therapy. When the cooling element is used, the cooling element lowers the temperature of the liquid in the liquid circulating channel and the cooling effect around the orbit of the eye socket is realized.

In an embodiment, the one or more stimulation element is a massager. The eye mask comprises a plurality of massage head on the convex edge at the periphery of the first depression cavity and the second depression cavity. The arrangement of the plurality of massage head allows massaging around the eye socket of the user.

In another embodiment of the present invention, the inner layer 102 further comprises a medicament storage chamber that stores a liquid medicament. A plurality of pores are provided in the inner layer 102 that are connected with the medicament storage chamber. A flexible button is provided on the top or front of the inner layer 102. When the user presses the flexile button, the button squeezes the medicament storage chamber to dispense the liquid medicament from the plurality of pores. The liquid medicament is dispensed around the eye socket. When used with micro-current therapy, the liquid medicament augment the therapy effect. The inner layer 102 may be provide with a medicament channel that circulates the liquid medicament from the medicament storage chamber to the plurality of pores.

The eye mask further comprises a battery 114 connected with a control circuit board 112. The control circuit board 112 is provided with a button 110 and a controller cover 108. The outer layer 104 is provided with a protrusion 140 accommodating the controller cover 108. The controller cover 108 is wrapped in the protrusion 140 on the outer layer 104. The outer layer has a certain softness, so that the user can press the button 110 comfortably.

The control circuit board 112 is provided with a charging part, and a corresponding charging port 142 is arranged on the protrusion 140.

In an embodiment of the present invention, the present invention provides a method for using the eye mask for providing therapy to a user's eyes. When worn by a user, the plurality of LEDs 116 provides phototherapy to the eyes. The micro-current element provides micro-current over the orbit of eyes and stimulate the soothing effect over the eyes. The method avoids direct contact of the plurality of LEDs 116 with eye socket as the plurality of LEDs 116 are arranged in the first depression cavity and the second depression cavity in the inner layer 102 of the eye mask. The direct exposure of eyes with the light is further prevented by the protective layer, in form of a spun fabric that allows light to pass through it.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. An eye mask for treating eyes, comprising:
an inner layer, the inner layer is provided with a first depression cavity corresponding to a left eye of a user and a second depression cavity corresponding to a right eye of the user;
an outer layer;
a circuit board arranged between the inner layer and the outer layer;
a plurality of LEDs arranged on the circuit board to target the left eye and the right eye of the user, such that the plurality of LEDs are positioned over the first depression cavity and the second depression cavity; wherein the first depression cavity and the second depression cavity creates a gap between the plurality of LEDs and eyes of the user; and
a protective layer arranged in the first depression cavity and the second depression cavity to protect the left eye and the right eye of the user from direct exposure to light emitted by the plurality of LEDs.

2. The eye mask of claim 1, wherein the first depression cavity and the second depression cavity are concave in the direction of the outer layer.

3. The eye mask of claim 1, wherein periphery of the first depression cavity and the second depression cavity forms a convex edge.

4. The eye mask of claim 3, wherein the convex edge comprises an embedded liquid flow channel to store a liquid around the convex edge.

5. The eye mask of claim 4, wherein the eye mask further comprises a heating element or a cooling element in contact with the liquid the embedded liquid channel to heat or cool the liquid.

6. The eye mask of claim 1, wherein the protective layer is made of a fabric or textile material.

7. The eye mask of claim 1, wherein the first depression cavity and the second depression cavity in the inner layer is provided with a plurality of holes for the plurality of LEDs to pass through the plurality of holes and the plurality of holes allow the plurality of LEDs to position over the first depression cavity and the second depression cavity.

8. The eye mask of claim 1 further comprising a plurality of massage head for providing massage therapy around the eyes of the user.

9. An eye mask for treating eyes, comprising:
an inner layer, the inner layer is provided with a first depression cavity corresponding to a left eye of a user and a second depression cavity corresponding to a right eye of the user;
an outer layer;
a circuit board arranged between the inner layer and the outer layer;
a plurality of LEDs arranged on the circuit board to target the left eye and the right eye of the user, such that the plurality of LEDs are positioned over the first depression cavity and the second depression cavity;
wherein the first depression cavity and the second depression cavity creates a gap between the plurality of LEDs and eyes of the user;
a micro-current element arranged between the inner layer and the circuit board, the micro-current element is electrically connected with the circuit board, wherein electrodes of the micro-current element are arranged on the periphery of the first depression cavity and the second depression cavity to provide micro-current at orbit around eyes of the user; and
a protective layer arranged in the first depression cavity and the second depression cavity to protect the left eye and the right eye of the user from direct exposure to light emitted by the plurality of LEDs.

10. The eye mask of claim 9, wherein the first depression cavity and the second depression cavity are concave in the direction of the outer layer and periphery of the first depression cavity and the second depression cavity forms a convex edge.

11. The eye mask of claim 10, wherein the convex edge comprises an embedded liquid flow channel to store a liquid around the convex edge.

12. The eye mask of claim 11, wherein the eye mask further comprises a heating element or a cooling element in contact with the liquid the embedded liquid channel to heat or cool the liquid.

13. The eye mask of claim 10, wherein the eye mask further comprises a plurality of massage head on the convex edge to provide massage therapy around the eyes of the user.

14. The eye mask of claim 9, wherein the micro-current comprises a conductive cloth and a conductive silica gel, the conductive silica gel is arranged on the conductive cloth.

15. The eye mask of claim 14, wherein the electrodes of the micro-current element is a conductive protrusion, the conductive protrusion is located on the conductive silica gel, and the conductive protrusion passes through a first through hole.

16. The eye mask of claim 9, wherein the protective layer is made of a fabric or textile material.

17. The eye mask of claim 9, wherein the first depression cavity and the second depression cavity in the inner layer is provided with a plurality of holes for the plurality of LEDs to pass through the plurality of holes.

18. The eye mask of claim 17, wherein the position of the plurality of LEDs on the circuit board is corresponding to the first depression cavity and the second depression cavity, and the plurality of holes allow the plurality of LEDs to positioned on the first depression cavity and the second depression cavity.

19. The eye mask of claim 10, wherein the convex edge further comprises a plurality of pores for dispensing a medicament over surface of eyes of the user.

20. A method for providing treatment to eyes, the method comprising:
providing a phototherapy to eyes by a plurality of LEDs positioned in an eye mask;
avoiding direct contact of the plurality of LEDs with eye socket by arranging the plurality of LEDs in a first depression cavity and a second depression cavity in an inner layer of the eye mask;
providing a protective layer in the first depression cavity and the second depression cavity to prevent direct exposure of light emitted by the plurality of LEDs with eyes of a user; and
providing a micro-current therapy to eyes of the user using a micro-current element provided in the eye mask.
